(19) United States
(12) Patent Application Publication
(10) Pub. No.: US 2012/0022407 A1
(43) Pub. Date: Jan. 26, 2012

(54) DEVICE FOR LOCATING A STRUCTURE INSIDE A BODY

(75) Inventor: Timotheus Joan Marie Lechner, Drunen (NL)

(73) Assignee: APAD Octrooi B.V., S-Hertogenbosch (NL)

(21) Appl. No.: 13/139,816

(22) PCT Filed: Dec. 17, 2009
(86) PCT No.: PCT/NL2009/000261

§ 371 Date: Sep. 9, 2011

(30) Foreign Application Priority Data
Dec. 18, 2008 (NL) 2002343

(51) Int. Cl. A61B 5/103

(52) U.S. Cl. 600/587

(57) ABSTRACT

A device for locating a structure (15) inside a body (10) includes a hollow needle (20), a liquid-filled reservoir (31) which is sealingly closed off at one end by means of a displaceable piston (32) and at the other end is in communication with the hollow needle; a sensor (40) for providing a pressure-measurement signal which is representative of the pressure prevailing in the liquid; a converter (50) for converting the pressure-measurement signal provided by the sensor into a converted signal suitable for further processing; and a display device (90) designed to emit at least one visual display signal which is a function of the converted signal. The device further includes a frame (200, 300) which can be worn on the head by a user and can be fixed on the head of the user, the visual display signal being in each case displayed in the range of vision of the user who is wearing the unit on his or her head in every position of the head of the user.

The present invention relates to a device for locating a structure inside a body by means of a hollow needle, comprising:
a sensor for providing a pressure-measurement signal which is representative of the pressure prevailing in the liquid which is present in a reservoir which can be connected to said hollow needle;
a converter for converting the pressure-measurement signal provided by the sensor into at least one converted signal which is suitable for further processing; and
a display device designed to emit at least one visual display signal in response to the at least one converted signal.

Such a device is known for example from EP 0,538,259. The device disclosed in EP 0,538,259 comprises a needle which is designed to be introduced into a body and an injection syringe which contains an isotonic liquid. The needle and the injection syringe are in communication with one another via a T-shaped connector. A pressure sensor is also connected to this T-shaped connector and serves to detect and measure the pressure prevailing in the liquid in the injection syringe. The pressure sensor is connected to a display device for displaying the pressure data in the form of numerical values. The known device furthermore comprises a converter for processing (converting) the data provided by the pressure sensor in order that the rate of the pressure variation as a result of a movement of a needle in the body can always be determined. The data provided by the converter and the pressure sensor are continuously compared with margins stored in the converter. Depending on the outcome of the comparisons, warning means may be activated.

The device disclosed in EP 0,538,259 has a number of drawbacks. Although the physician can see the instantaneous pressure data on a viewing screen during the introduction of the needle into the body of a patient, the physician will have to look at said viewing screen for this purpose. This means that the physician has to avert his eyes from the location where the needle is introduced into the body of the patient, which is undesirable. The introduction of a needle into the body of a patient is an operation which requires a high degree of accuracy and is susceptible to errors. One example thereof is the so-called epidural which is often used for administering anaesthetics. In this case, the needle is pushed through the skin on the back of the patient, through the tissue situated between the vertebrae and eventually has to stop in the epidural space which is situated in front of the vertebrae in order to introduce the anaesthetic. However, if the needle pushes through too far, it ends up in the tissue which is situated behind the epidural space, such as the dura mater, nerve bundles, etc., which can lead to severe disablement. It is also known to extract liquid from the body from intracorporeal spaces by means of a hollow needle, for example for closer inspection or other purposes. In all these cases, it is important that the tip of the hollow needle is accurately introduced into the respective structure.

Accurate positioning of the tip of the hollow needle is difficult, as the circumstances differ from patient to patient. The insertion depth of the needle is affected by many factors, such as age, obesity, sex, differences in tissue layer thickness, etc. It is thus important, on the one hand, that the practitioner—who in the context of the present application is understood to be a physician or other person—can concentrate fully on the location where the operation takes place in order to be able to manipulate the needle very accurately and that the practitioner, on the other hand, has information with respect to the location of the tip of the hollow needle in the tissue or the kind of tissue in which the hollow needle is situated.

With regard to supplying the practitioner with information about the location of the needle tip, it is known, inter alia from said EP 538,259, to use the pressure or changes in pressure of the liquid in the hollow needle for this purpose. This information is made available on a viewing screen which is provided in a location at a distance from the location where the operation is taking place. It is also known to make this information available in the form of a sound signal. Consideration may for example be given to a sound signal whose intensity depends on the detected pressure or pressure variation. The advantage of a sound signal is that the practitioner can concentrate fully on the location where the operation is taking place and can therefore focus his eyes completely on the spot where the needle enters the body. A drawback of a sound signal is that it is also heard by the patient who is generally conscious. The patient, who is usually already quite tense, tends to react to the sound signal, for example by moving or by becoming more tense, particularly when the sound signal changes, since the patient, either consciously or subconsciously, realises that a change in the sound signal means something. Moreover, in many cases it has already been explained to the patient beforehand what the change in the sound signal means. The fact that the patient is tense results in tension in the tissue. Such tension and movements make it even more difficult to position the tip of the hollow needle accurately and to keep it positioned accurately.

US 2006/149,161 describes a measuring device for measuring an increase in the pressure of the CSF (=CerebroSpinal Fluid) while the CSF is being tapped from the back of a patient, as an increase in the pressure of the CSF is an indication of hydrocephalus. Measuring the pressure of the CSF thus aids in diagnosing hydrocephalus and treating hydrocephalus is made possible by tapping CSF. The measuring device from US 2006/149,161 is to this end fitted with a sensor for providing a pressure-measurement signal, a converter for converting the pressure-measurement signal to a further signal and also a display for emitting at least one visual display signal. The display is provided here in the form of a separate, hand-held instrument which is connected to the patient via a needle and hoses and remains with the patient during use. In use, a connection to the CSF of the patient is made first by introducing a needle into the patient and connecting the hoses required for tapping CSF. Only then is the measuring device from US 2006/149,161 connected to the CSF system of the patient. Because the hand-held instrument with display from US 2006/149,161 is at some distance from the patient, the connecting hoses (see US-2006/0149,161, §0019) are filled with saline. Similar to EP 538,259, those who wish to read the display have to keep their eyes on a display which is arranged at a great distance from the location where the needle has already been introduced into the patient.

It is an object of the present invention to provide an improved device for locating a structure, such as a cavity, space or specific tissue structure, inside a body by means of a hollow needle.

This object is achieved according to the invention by using a so-called “head-up display” or HUD for short. A HUD is a system which displays visual information, for example by projection or other means, within the range of vision of the user of the HUD and more particularly in such a way that the visual information is in the range of vision of the user in substantially any position of the head of the user. In this context, the term range of vision is understood to mean the area which a person can perceive with his eyes, with the proviso that the eyes can move if required.

The object of the invention is thus achieved by providing a device for locating a cavity inside a body by means of a hollow needle, comprising:
a sensor for providing a pressure-measurement signal which is representative of the pressure prevailing in a liquid which is present in a reservoir which can be connected to said hollow needle;
a converter for converting the pressure-measurement signal provided by the sensor into at least one converted signal which is suitable for further processing; and
a display device designed to emit at least one visual display signal in response to the at least one converted signal;
   the invention being characterized by the fact
that the display device forms part of a system which, irrespective of the position of the head of the user, is designed to display the at least one visual display signal in the range of vision of the user, the range of vision of the user being defined as an area which, on the one hand, is fixed with respect to the head of the user and, on the other hand, can be perceived by the eyes of the user of the system;
   and/or
that the device furthermore comprises a frame which can be used on the head by a user, the display device being arranged on the frame in such a manner that the at least one visual display signal which is to be emitted can be perceived by the eyes of the user who is wearing the frame on his or her head.

The sensor for providing the pressure-measurement signal is in this case connected (wirelessly, by wire or otherwise) or can at least be connected to the converter in order to pass the pressure-measurement signal on to the converter. The converter is in this case connected—optionally by means of a further converter, processing device or otherwise—(wirelessly, by wire or otherwise) or can at least be connected to the display device in order to pass the converted signal to the display device.

The practitioner can thus, on the one hand, visually concentrate completely on the patient, in particular on the spot where the needle enters the patient, and, on the other hand, receive visual information about the pressure and/or pressure variations of the liquid in the hollow needle without having to turn his head away for this purpose. This visual information is presented within the range of vision of the practitioner, irrespective of the position of the head. All this takes place in real time during the operation itself and the information is directly related to the operation and specific to the position of the tip of the needle in the body of the patient. Moreover, this information can hardly be detected by the patient, if at all, and can easily be kept from the patient completely, if desired. The patient is thus not influenced by this information.

According to a specific embodiment of the invention, the at least one visual display signal comprises an instantaneous signal which is indicative of the instantaneous pressure of the liquid. The practitioner thus has an idea of the pressure level and can also observe changes in said pressure level. Such an instantaneous signal can for example also be an image of a numerical figure. According to a further embodiment, it is advantageous in this case if the display device comprises an illuminable line, the illuminated length of which can vary as a function of the instantaneous signal, and if the illuminable line is provided in the range of vision. Such an illuminable line, which may, for example, be composed of a series of a plurality of light elements, such as LEDs, can easily be produced. If the pressure value detected by the pressure sensor increases, a greater number of light elements will light up and if the pressure detected by the pressure sensor decreases, a smaller number of light elements will light up. Thus, the length of adjacent light elements which light up varies depending on the measurement signal detected by the pressure sensor. It will be clear that the relationship between the length which lights up and the detected pressure can also be the reverse, in the sense that a large number of light elements lighting up indicates a lower pressure while a smaller number of light elements lighting up indicates a higher pressure.

According to a further embodiment, the at least one visual display signal comprises a progress signal which indicates the progress over time of the pressure which prevails in the liquid. Consideration may, for example, be given to a representation in the form of a graph showing the pressure level in the vertical direction and the time in the horizontal direction. The practitioner will then always be able to see the instantaneous pressure as well as the preceding pressure values and will thus have an idea of the course thereof.

According to a further embodiment, the display device comprises a projector which is designed to project said at least one visual display signal in the range of vision of the user who is wearing the unit on his or her head. If the display device is provided on a frame which is worn on the head, the projector will also be arranged on the frame. This projector can display the projection image, which thus forms a visual display signal, on diverse projection surfaces. The projection surface may, for example, be the skin of the patient or a surface provided on this skin. Thus, the projection image may be situated on, next to or around the location where the needle is inserted into the skin. According to a further embodiment, the display device in this case furthermore comprises a projection surface. The projection surface may, for example, be an element which is arranged on the skin of the patient or on the clothing of the patient or on a surgical blanket/cloth provided on the patient at the location of the operation. However, the projection surface can also be provided on the frame, with the projector being directed at that projection surface. Thus, there is a fixed relationship between the projector and the projection surface.

According to a further embodiment of the invention, the converter is designed to emit a switching signal to the display device when the progress of the pressure-measurement signal shows a change in pressure of a predetermined magnitude, and the display device is designed to emit a warning signal in the form of a visual display signal in response to the switching signal. Thus, the alertness of the practitioner can be increased at the moment when a change in pressure occurs, since a change in pressure indicates that the tip of the hollow needle is passing from one tissue structure to another tissue structure or cavity and the practitioner is thus warned.

According to yet a further embodiment, the device furthermore comprises automatic drive means for exerting a force on the liquid. In this way, the liquid is automatically kept under pressure, for example by means of a pump, and the practitioner does not have to do this himself. The practitioner can thus concentrate fully on positioning the tip of the hollow needle. A further advantage is the fact that automatic drive means are able to maintain the pressure at a very constant level. If the pressure is maintained manually, for example by operating a piston of an injection syringe or by squeezing a balloon, pressure variations will also result due to variations in muscle power. The sensor is in this case designed specifically to register the force exerted by the drive means on the liquid. The sensor does therefore not have to measure the liquid pressure directly but can record the force exerted by the drive means.

According to a further embodiment, the converter is designed in particular to emit the converted signal in the form of an electric voltage. For the purpose of recording, it is advantageous according to the invention if the device furthermore comprises recording means for recording the progress of the pressure-measurement signal over the course of time.

According to a further embodiment, the device furthermore comprises said hollow needle.

According to yet a further embodiment, the device furthermore comprises said liquid reservoir which, on the one hand, can be connected to pressure means for pressurizing said liquid and, on the other hand, can be connected to said hollow needle.

According to yet a further development, the reservoir is filled with said liquid. Said liquid is preferably an isotonic liquid.

Said aspects and other aspects, characteristic features and advantages of the present invention will be explained in more detail by means of the following description or preferred embodiments of a locating device according to the invention with reference to the drawing, in which identical parts are denoted by the same reference numerals, and in which:

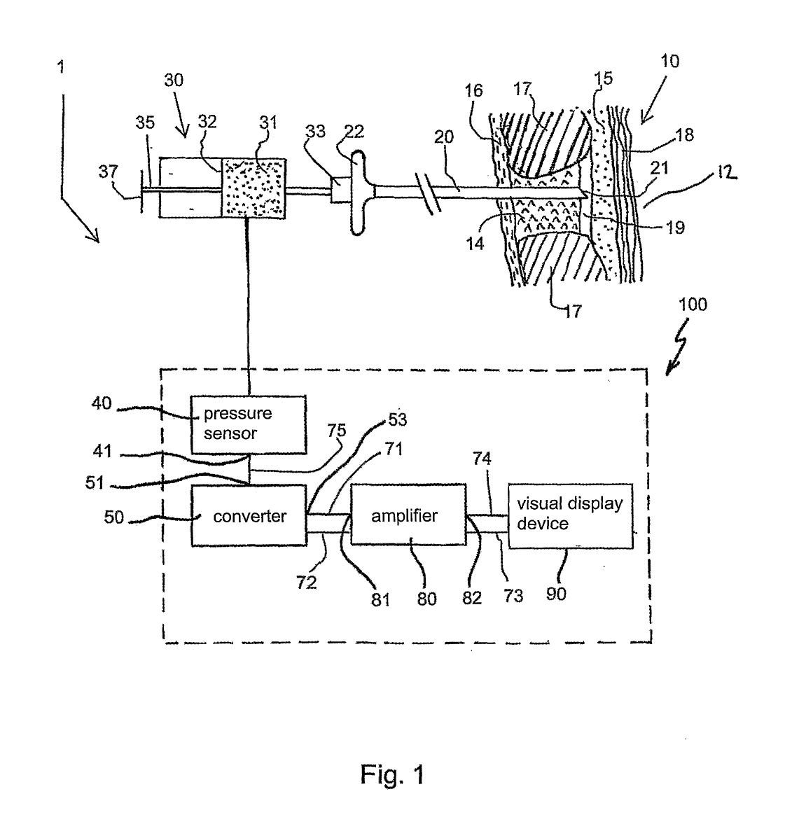

FIG. 1 shows a diagrammatic view of a first preferred embodiment of the locating device according to the invention, in which a portion of said locating device is shown diagrammatically;

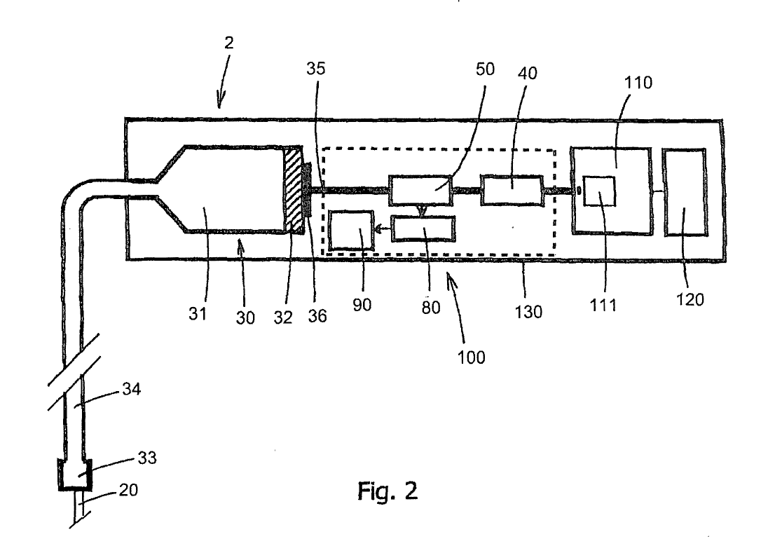

FIG. 2 shows a diagrammatic view of a portion of a second preferred embodiment of the locating device according to the invention;

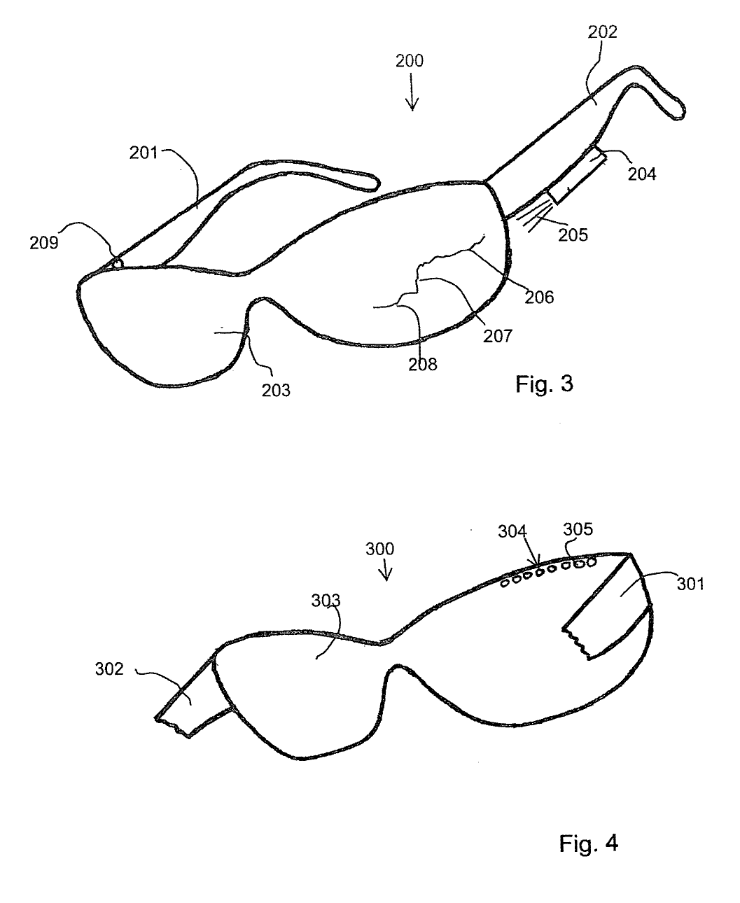

FIG. 3 shows a diagrammatic, perspective view of a first frame with a display device according to the invention; and

FIG. 4 shows a diagrammatic, perspective view of a second frame with a display device according to the invention.

FIG. 1 shows a first preferred embodiment of the locating device according to the invention, which is denoted overall by reference numeral 1.

FIG. 1 shows one of the possible uses of the locating device 1, i.e. the use in locating the epidural space in a human body. A small portion of the human body is illustrated in cross section in FIG. 1 and is denoted overall by reference numeral 10. The epidural space is denoted by reference numeral 15. Reference numeral 16 denotes the skin, reference numeral 17 denotes a protrusion of a vertebra, reference numeral 14 the ligamentum interspinosum, reference numeral 19 the ligamentum flatum, and reference numeral 18 denotes the dura mater (the hard meninx fibrosa), and 12 the backbone.

The locating device 1 comprises a hollow puncture needle 20 with a needle tip 21. In the example shown in FIG. 1, the puncture needle 20 has been introduced part way into the body 10 and the needle tip 21 is situated in the vicinity of the ligamentum flatum 19, just outside the epidural space 15. The puncture needle 20 is provided with a handle 22, which can be held by the person who is handling the puncture needle 20, for example a physician. This handle 22 enables the physician to exert a pushing or pulling force on the puncture needle 20, in order for it to be moved in a desired direction.

An injection syringe 30 is positioned in line with the puncture needle 20. The injection syringe 30 comprises a liquid-filled reservoir 31 and a displaceable piston 32 which closes off the said reservoir 31 in a sealed manner on one side. The piston 32 can be operated manually by means of a piston rod 35 and a button 37. The reservoir 31 is in communication with the puncture needle 20 via a connection part 33, it being possible for liquid to flow out of said reservoir 31 into said puncture needle 20. The liquid in the reservoir 31 is a sterile, isotonic liquid.

To measure the pressure in the liquid in the reservoir 31, the locating device 1 is provided with a pressure sensor 40 which is known per se. An output 41 of the pressure sensor 40 is connected to an input 51 of a converter 50, which is able to convert a pressure-measurement signal 75 provided by the pressure sensor 40 into at least one signal 71, 72 which can be used for further processing, for example an electric voltage. It should be noted that the pressure sensor 40 and the converter 50 can form a single unit instead of two separate elements.

One output 53 of the converter is connected to an input 81 of an amplifier 80 via a first signal conductor 71 for a first converted signal. A further output of the converter is connected to a further input of the amplifier 80 via a second signal conductor 72 for a second converted signal. The amplifier is designed to emit an amplified first converted signal to a third signal line 73 on an output 82 and to emit an amplified second converted signal to a fourth signal line 74 at another output. The third signal line 73 and the fourth signal line 74 lead to a visual display device 90 which is designed to emit two visual display signals. The amplifier can also be absent. Furthermore, the converter can also emit a single converted signal (instead of two converted signals) or even more than two converted signals.

The assembly comprising pressure sensor 40, converter 50, amplifier 80 and visual display device 90 is referred to below as the measuring and display unit 100, as shown by a dashed line in FIG. 1.

Before the locating device 1 can be used to locate an epidural space 15 in a body 10, it is preferable for the puncture needle 20 to have been introduced into the body 10 first. Then, the injection syringe 30 and the measuring and display unit 100 coupled thereto can be connected to the puncture needle 20.

If the person who is handling the injection syringe 30 and the puncture needle 20, for example a practitioner, exerts pressure on the piston 32, pressure will be built up in the liquid which is situated in the reservoir 31. As a result of this pressure, the liquid will tend to flow out of the unit comprising reservoir 31 and puncture needle 20 via the needle tip 21. In response to this effect of the pressure on the plunger 32, the liquid at the needle tip 21 is subject to a counterpressure as a result of the resistance to absorption of the liquid by the tissue in which the needle tip 21 is situated. If the practitioner keeps the pressure on the plunger 32 at a defined level while he is moving the puncture needle 20 in the direction of the epidural space 15, the pressure in the liquid in the reservoir 31 is always a measure of the pressure state at the needle tip 21.

On the basis of the pressure in the liquid, the pressure sensor 40 continuously emits, via the output 41, a pressure-measurement signal to the input 51 of the converter 50. The pressure-measurement signal is converted by the converter 50 into a signal which can at least be used for further processing, in this example an electric voltage. The pressure-measurement signal is supplied as voltage signal via one or more signal lines 71, 72 to the amplifier 80 or directly to the visual display device 90. In this embodiment, the continuous pressure-measurement signal is processed to form, on the one hand, a continuous converted signal which is provided to the amplifier 89 via the signal line 71 (or directly to the visual display device 90) and, on the other hand, a switching signal which is provided to the amplifier via signal line 72. The continuous signal is representative of the instantaneous pressure prevailing in the liquid and makes it possible for the display device to emit an instantaneous signal which is indicative of the instantaneous pressure prevailing in the liquid. The switching signal may be a signal which either has a first value or a second value and makes it possible for the display device to emit a warning signal. Thus, it is for example possible to emit a warning signal if the pressure-measurement signal shows a change in pressure which is greater than a predetermined value per unit time. When the needle tip 21 penetrates another tissue, it will either be easier or harder for the liquid to flow out of the needle. The liquid in the reservoir will therefore experience a change in pressure. For example, when the epidural space 15 is reached, the liquid will experience a drop in pressure. The converter is able to detect changes in the pressure-measurement signal and to emit a switching signal—by for example changing from a value of zero to a value greater than zero—if the change in the pressure-measurement signal exceeds a predetermined threshold value. The visual display device will emit a visual warning signal in response to the switching signal.

Any changes in the pressure which occur during a displacement of the needle tip 21 lead to changes in the instantaneous signal emitted by the display device. The practitioner who is handling the puncture needle 20 can use any changes in the instantaneous signal which he detects over the course of time to determine what changes are occurring in the pressure. In view of the knowledge that a relatively great pressure drop occurs when the epidural space 15 is reached, the practitioner can infer from a sudden and significant change in the instantaneous signal that the needle tip 21 has reached the epidural space 15 and that he must stop moving the puncture needle 21 into the body 10. To this end, the practitioner has to focus on the instantaneous signal. By using the aforementioned switching signal, the practitioner does not have to concentrate so much on the instantaneous signal since a warning signal will warn him if the pressure drop is greater than a predefined and predetermined value. The warning signal then alerts the practitioner to the fact that he now needs to concentrate on the instantaneous signal. When the switching signal and the visual warning signal in response thereto are used, it is also possible not to use the instantaneous signal. The instantaneous signal and the associated instantaneous converted signal can thus also be omitted/be absent.

When using the locating device 1, calibration of the pressure-measurement signal is not critical, since the practitioner is working on the basis of changes which he detects in the indicative aspect or aspects of the visual display signal, the absolute value of the indicative aspect or aspects of the visual display signal being of subordinate importance.

The display device 90 may, for example, be designed to reproduce the visual signal in the form of numerical values. If desired, means may in this case be provided which allow the numerical values to be calibrated, but this is not imperative, since the practitioner can rely on the changes in the visual signal to determine whether or not the needle tip 21 has reached the epidural cavity 15.

FIG. 2 diagrammatically depicts a portion of a second preferred embodiment of the locating device according to the invention, which is denoted overall by reference numeral 2. The second embodiment of the locating device according to the invention comprises the same components as the first preferred embodiment shown in FIG. 1.

The hollow puncture needle 20 which forms part of the locating device 2 is only partially shown in FIG. 2. The reservoir 31 of the injection syringe 30 is connected to the puncture needle 20 by means of a hose 34 and the connection part 33.

The locating device 2 is designed to automatically—that is to say not manually—exert pressure on the piston 32 of the reservoir 31. For this purpose, the locating device 2 comprises a connecting rod 35, one end of which is in communication with a drive unit 110 and the other end of which is provided with a pressure disc 36. The drive unit 110 is used to exert a force on the connecting rod 35 in order to effect a movement in the axial direction of the connecting rod 35. The pressure disc 36 is intended to bear against a significant part of the surface of the plunger 32. In this example, the drive unit 110 is connected to a (storage) battery 120. Other possible ways of supplying energy to the drive unit 110 are also conceivable. For example, the locating device 2 could be provided with means for connecting the drive unit 110 to the mains.

A significant advantage of the automatic application of pressure to the piston 32 is that the practitioner who is using the locating device 2 can use both hands to move the puncture needle. As has already been stated in the description of the first preferred embodiment of the locating device, the pressure state at the needle tip is continuously rendered visible by means of the visual display signal emitted by the display device. Consequently, it is not necessary for the practitioner to exert the pressure on the piston 32 himself.

The injection syringe 30, the connecting rod 35, the drive unit 110 and the (storage) battery 120 are all arranged in the interior of a housing 130, as is the measuring and display unit 100, possibly with the exception of the visual display device 90. An arrangement of this type is preferred, since it results in a compact assembly which can easily be connected to the puncture needle 20.

The injection syringe 30 is accommodated in a cavity which is created for this purpose in the housing 130, which at the location of the said cavity is preferably designed in such a manner that an injection syringe 30 can easily be placed into the housing 130 before use and can easily be removed again from the housing 130 after use.

Clamping means (not shown) may be provided on the exterior of the housing 130, in order to allow the said housing 130 to be clamped to any desired object, such as for example a table or a bed.

In this example, the pressure sensor 40 comprises a force pick-up which is designed to pick up the force exerted on the connecting rod 35 by the drive unit 110. In this case, the converter 50 is designed as a force-voltage converter. The drive unit 110 comprises a control unit 111 which is in communication with the force pick-up of the pressure sensor 40.

By way of example, a portion of the connecting rod 35 may be provided with helical toothing, in which case the drive unit 110 is provided with a toothed wheel which meshes with the said portion. The force pick-up of the pressure sensor 40 is in this case designed to measure the force exerted on the connecting rod 35 by the wheel.

The locating device 2 is intended to be used for locating an internal structure, such as for example the epidural space in a human body. For an application of this type, the puncture needle is moved into the body until the needle tip has reached the epidural space. The measuring and display unit 100 of the locating device 2 is used in the process to determine the instantaneous pressure state at the needle tip and to reproduce said pressure state visually by means of continuous and/or discontinuous signals.

The process of locating the epidural space begins with bringing the isotonic liquid in the reservoir 31 up to a defined initial pressure. For this purpose, the drive unit 110 exerts force on the connecting rod 35, the pressure disc 36 of which bears against the piston 32. As a result of the force exerted, the connecting rod 35 will move in the axial direction and the piston 32 will likewise move in the direction of the liquid in the reservoir 31. In the process, the liquid tends to move from the reservoir 31 and the puncture needle towards the body, and in the process is subject to a counterpressure which is exerted by the body. An increase in the pressure of the liquid will occur under the influence of the pressure exerted on the liquid by the plunger 32 and the counterpressure exerted on the liquid by the body. The force which is exerted on the connecting rod 35 by the drive unit 110 is directly related to the pressure state at the needle tip. The constant character of the force can in this case easily be detected, since the visual signal emitted by the display device 90 is directly related to the force and/or to changes in the force.

The drive unit 110 is designed to move the connecting rod 35 at a defined, constant speed when the liquid in the reservoir 31 is at the starting pressure. The force which is exerted on the connecting rod 35 by the drive unit 110 is directly related to the pressure state at the needle tip.

When the needle tip reaches the epidural cavity, the counterpressure will drop suddenly and significantly, and the pressure prevailing in the liquid will likewise drop suddenly and significantly. At that time, the drive unit 110 has to exert in relative terms a much lower force in order to move the connecting rod 35 than was the case before the epidural space was reached. The sudden and significant change in the force is observed by the practitioner as a sudden and significant change in the visual display signal. The practitioner can infer from a change of this type that the needle tip has reached the epidural space and that he must stop moving the puncture needle. He can then disconnect the connection between the puncture needle and the remaining components of the locating device at the connection part 33, in order for an injection syringe containing the substances which are to be introduced into the epidural space to be placed onto the puncture needle or in order to introduce what is known as an epidural catheter.

In a preferred embodiment which is not shown, the locating device according to the invention comprises recording means for recording the profile of the pressure-measurement signal over the course of time.

According to the invention, a so-called ‘head-up display’, or HUD for short, is used. An HUD is a system in which visual information which usually has to be read from an instrument is displayed directly, for example by means of projection or otherwise, in the range of vision of the user of the HUD. If the expression ‘range of vision of a person’ is understood to mean the area which said person can see with their eyes without said person moving their head and it is recognised that this area moves along proportionally as the person moves their head, then the range of vision can be defined according to the invention as the area which is fixed with respect to the head and can be perceived by the eyes. According to the invention, an HUD system is therefore understood to mean a system which displays visual information in the range of vision of the user, that is to say that area which is fixed with respect to the head and can be perceived by the eyes of the user of the HUD system. Aeroplane and helicopter pilots are known to use HUD systems in which data are projected onto the visor provided on the pilot's helmet by means of a projector fitted in the helmet.

To this end, the device according to the invention comprises in particular a frame which can be worn on the head by the user and on which the display device is arranged in such a manner that, in each position of the head, the at least one visual display signal which is to be emitted is in each case displayed in the range of vision—that is to say the area fixed with respect to the head—which can be perceived by the eyes of the user who is wearing the frame on his or her head. According to the invention, the frame may comprise, for example, glasses or a helmet.

FIGS. 3 and 4 show two HUDs according to the invention which are designed as glasses.

FIG. 3 shows a frame in the form of glasses 200 which can be worn on the head and which are shown in a perspective front view, viewed counter to the viewing direction of the user of the glasses.

The glasses 200 comprise two arms 201 and 202 and a screen 203 which is transparent to the eye, also referred to as glass, although the screen 203 can also be made of a material other than glass, such as plastic.

On the arm 202 of the glasses, a projector 204 is provided which emits a light beam 205. This light beam 205 produces a projection in the form of a graphic signal 206, 207, 208 showing the profile of the pressure in the liquid over the course of time on the transparent screen. By way of example, this illustration shows a projection in which the pressure initially has a relatively high value 206, then experiences a pressure drop, at 207, and subsequently has a relatively low value 208 compared to the previous high value 206. The pressure drop 207 indicates that the tip 21 of the hollow needle 20 has reached a tissue, such as the epidural space to be located, which has a lower resistance to the liquid pressure. It should be noted that it is also possible to allow the light beam 205 to pass through the screen in order to produce a projection on an object other than the screen. This other object may, for example, be the skin of the patient at the location where the needle was introduced into the patient.

On the inside of the arm 201 of the glasses, in the vicinity of the screen 203, a light element 209, such as an LED, is provided. The converter may be designed such that it emits a control signal in order to switch this light element on when a pressure drop or pressure increase of a predetermined magnitude, such as the pressure drop 207, occurs. Despite the fact that the light element 209 may possibly be on the edge of or just outside the range of vision, the light emitted by the light element will easily be perceived by the eye. Thus, the practitioner can be warned of a certain pressure drop/pressure increase having occurred, so that the practitioner follows the signal 206, 207, 208 more closely. It is also conceivable for the projector 205 and the image projected thereby to be absent and for the practitioner to only make use of the warning signal emitted by the light element 205. It is likewise conceivable to design the converter in such a way that the signal it emits causes the light element 209 to flash at a frequency depending on the pressure detected by the sensor. The flashing frequency then provides the practitioner with information about the pressure level and any changes therein. Instead of on an arm, the light element may also be fitted on the screen 203. Furthermore, it is also possible for several light elements 209 to be provided.

FIG. 4 shows different glasses 300, the arms 301 and 302 of which have only been partially illustrated. Here, the view is again perspective, but now seen from the eyes of the wearer. These glasses 300 are provided with a line of lights 304 which is arranged on the screen. The line of lights 305 can light up and the length of the lit-up portion thereof is indicative of the value of the pressure detected by the sensor. The line of lights can be designed in a number of different ways which are known per se. In this example, the line of lights 304 is composed of a series of light elements 305, for example LEDs. In this case, the number of light elements 305 is eight, but this number may also be lower or higher. Alternatively, the line of lights can also be driven in accordance with the variants which have been described for light element 209.

It will be clear to the person skilled in the art that the scope of the present invention is not restricted to the examples which have been discussed above, but rather that various changes and modifications to these examples are possible without departing from the scope of the invention as defined in the appended claims.

The above text has described an application of the locating device according to the invention for locating an epidural cavity 15 in a human body 10. This does not detract from the fact that the locating device can be used to locate any desired structure in any desired body, provided that pressure variations which occur in the liquid during displacement of the puncture needle in a portion of the body which precedes the structure to be located are significantly smaller than pressure variations which occur in the liquid when the needle tip reaches the structure.

According to yet another embodiment of the invention, the device according to the invention comprises a frame which can be worn on the head—such as a helmet, glasses or a headband—on which a projector is provided. This frame can be worn on the head in such a manner that the projector is or can at least be directed in the viewing direction of the head of the user in order to project a visual display signal at a distance greater than 20 cm, for example approximately 30-70 cm. If desired, the projector can to this end be adjusted with respect to the frame in order to modify the direction of projection. Thus, the visual display signal can be projected at the location of the operation (usually the spot where the needle is introduced into the patient) or near said location. The practitioner can then see the projection without having to avert his focus from the location of the operation.

The present invention can be described in more detail by means of the following clauses:

1. Device for locating a structure 15 inside a body 10 by means of a hollow needle 20, comprising:
a sensor 40 for providing a pressure-measurement signal which is representative of the pressure prevailing in a liquid which is present in a reservoir 31 which can be connected to said hollow needle 20;
a converter 50 for converting the pressure-measurement signal provided by the sensor 40 into at least one converted signal which is suitable for further processing; and
a display device designed to emit at least one visual display signal in response to the at least one converted signal;
   characterized in that the display device forms part of a system which displays the at least one visual display signal in the area which is fixed with respect to the head of the user and can be perceived by the eyes of the user of the system.
   2. Device for locating a structure 15 inside a body 10 by means of a hollow needle 20, comprising:
a sensor 40 for providing a pressure-measurement signal which is representative of the pressure prevailing in a liquid which is present in a reservoir 31 which can be connected to said hollow needle 20;
a converter 50 for converting the pressure-measurement signal provided by the sensor 40 into at least one converted signal which is suitable for further processing; and
a display device designed to emit at least one visual display signal in response to the at least one converted signal;
   characterized in that the display device forms part of a system which, irrespective of the position of the head of the user, is designed to display at least one visual display signal in the range of vision of the user, the range of vision of the user being defined as an area which, on the one hand, is fixed with respect to the head of the user and, on the other hand, can be perceived by the eyes of the user of the system.
   3. Device according to clause 1 or 2, in which the system furthermore comprises a frame which can be worn on the head by a user, the display device being arranged on the frame in such a manner that the at least one visual display signal which is to be emitted can be perceived by the eyes of the user who is wearing the frame on his or her head.
   4. Device for locating a structure 15 inside a body 10 by means of a hollow needle 20, comprising:
a sensor 40 for providing a pressure-measurement signal which is representative of the pressure prevailing in a liquid which is present in a reservoir 31 which can be connected to said hollow needle 20;
a converter 50 for converting the pressure-measurement signal provided by the sensor 40 into at least one converted signal which is suitable for further processing; and
a display device designed to emit at least one visual display signal in response to the at least one converted signal;
   characterized in that the device furthermore comprises a frame which can be worn on the head by a user, the display device being fitted on the frame in such a manner that the at least one visual display signal which is to be emitted can be perceived by the eyes of the user who is wearing the frame on his or her head.
   5. Device according to one of the preceding clauses, in which the at least one visual display signal comprises an instantaneous signal which is indicative of the instantaneous pressure prevailing in the liquid.
   6. Device according to clause 5, in which the display device comprises an illuminable line, the illuminated length of which can vary as a function of the instantaneous signal, and in which the illuminable line is provided in the range of vision.
   7. Device according to one of the preceding clauses, in which the at least one visual display signal comprises a progress signal which is indicative of the progress over time of the pressure prevailing in the liquid.
   8. Device according to one of the preceding clauses, in which the display device comprises a projector which, to the extent that it is dependent on clause 3 or clause 4, is preferably provided on the frame and is designed to project said at least one visual display signal in the range of vision of the user who is preferably wearing the unit on his head.
   9. Device according to clause 8, in which the display device furthermore comprises a projection surface.
   10. Device according to clause 9, in which the projection surface is provided on the frame, and in which the projector is directed at the projection surface.
   11. Device according to one of the preceding clauses, in which the system comprises glasses which comprise two arms and a screen which is transparent to the eye.
   12. Device according to one of the preceding clauses, in which the converter is designed to emit a switching signal to the display device when the progress of the pressure-measurement signal shows a change in pressure of a predetermined magnitude, and in which the display device is designed to emit a warning signal in the form of a visual display signal in response to the switching signal.
   13. Device according to one of the preceding clauses, in which the device furthermore comprises automatic drive means 35, 110 for exerting a force on the liquid.
   14. Device according to clause 13, in which the sensor 40 is designed to register the force exerted on the liquid by the drive means 35, 110.
   15. Device according to one of the preceding clauses, in which the converter 50 is designed to emit the converted signal in the form of an electric voltage.
   16. Device according to one of the preceding clauses, in which the device furthermore comprises recording means for registering the progress of the pressure-measurement signal over the course of time.
   17. Device according to one of the preceding clauses, furthermore comprising said hollow needle 20.
   18. Device according to one of the preceding clauses, furthermore comprising a reservoir 31 which can be filled with said liquid and, on the one hand, can be connected to pressure means for pressurizing said liquid and, on the other hand, can be connected to said hollow needle 20.
   19. Device according to clause 18, in which the reservoir 31 is filled with said liquid, said liquid preferably being an isotonic liquid.

1. A device for locating a structure inside a body by means of a hollow needle, comprising:
a sensor for providing a pressure-measurement signal which is representative of the pressure prevailing in a liquid which is present in a reservoir which can be connected to said hollow needle;
a converter for converting the pressure-measurement signal provided by the sensor into at least one converted signal which is suitable for further processing; and
a display device designed to emit at least one visual display signal in response to the at least one converted signal;
wherein the display device forms part of a system which, irrespective of the position of the head of the user, is designed to display the at least one visual display signal in the range of vision of the user, the range of vision of said user being defined as an area which, on the one hand, is fixed with respect to the head of said user and, on the other hand, can be perceived by the eyes of said user of the system.

2. The Device according to claim 1, wherein the system comprises a frame which can be worn on the head by a user, the display device being arranged on the frame in such a manner that the at least one visual display signal which is to be emitted can be perceived by the eyes of the user who is wearing the frame on his or her head.

3. The Device according to claim 1, wherein the at least one visual display signal comprises an instantaneous signal which is indicative of the instantaneous pressure prevailing in the liquid.

4. The Device according to claim 3, wherein the display device comprises an illuminable line, the illuminated length of which can vary as a function of the instantaneous signal, and wherein the illuminable line is provided in the range of vision.

5. The Device according to claim 1, wherein the at least one visual display signal comprises a progress signal which is indicative of the progress over time of the pressure prevailing in the liquid.

6. The Device according to claim 2, wherein the display device comprises a projector which is provided on the frame and is designed to project said at least one visual display signal in the range of vision.

7. The Device according to claim 6, wherein the display device further comprises a projection surface.

8. The Device according to claim 7, wherein the projection surface is provided on the frame, and wherein the projector is directed at the projection surface.

9. The Device according to claim 1, wherein the system comprises glasses which comprise two arms and a screen which is transparent to the eye.

10. The Device according to claim 1, wherein the converter is designed to emit a switching signal to the display device when the progress of the pressure-measurement signal shows a change in pressure of a predetermined magnitude, and wherein the display device is designed to emit a warning signal in the form of a visual display signal in response to the switching signal.

11. The Device according to claim 10, wherein the sensor is designed to register the force exerted on the liquid by the drive means.

12. The Device according to claim 1, wherein the converter is designed to emit the converted signal in the form of an electric voltage.

13. The Device according to claim 1, wherein the device further comprises:
recording means for registering the progress of the pressure-measurement signal over the course of time; and/or
said hollow needle; and/or
automatic drive means for exerting force on the liquid.

14. The Device according to claim 1, further comprising a reservoir which can be filled with said liquid and, on the one hand, can be connected to pressure means for pressurizing said liquid and, on the other hand, can be connected to said hollow needle.

15. The Device according to claim 14, wherein the reservoir is filled with said liquid.

16. The Device according to claim 15, wherein said liquid is an isotonic liquid.
